(19) United States
(12) Patent Application Publication
(10) Pub. No.: US 2026/0355147 A1
(43) Pub. Date: Nov. 12, 2026

(54) COMPOSITIONS COMPRISING TRIGONELLINE AND A UROLITHIN

(71) Applicant: Amazentis SA, Lausanne (CH)

(72) Inventors: Christopher L. Rinsch, Lausanne (CH); Anurag Singh, Lausanne (CH); Davide D'Amico, Renens (CH); Andréane Fouassier, Prangins (CH)

(21) Appl. No.: 19/487,301

(22) PCT Filed: May 28, 2024
(86) PCT No.: PCT/IB2024/000256

§ 371 Date: Nov. 24, 2025

Related U.S. Application Data
(60) Provisional application No. 63/469,244, filed on May 26, 2023.

(51) Int. Cl. A61K 31/37; A61K 31/455; A61P 3/00

(52) U.S. Cl. CPC A61K 31/37; A61K 31/455; A61P 3/00

(57) ABSTRACT

Disclosed are compositions comprising a urolithin or a pharmaceutically acceptable salt thereof and trigonelline or a pharmaceutically acceptable salt thereof, and methods for use in increasing or maintaining mitochondrial function.

### RELATED APPLICATION

This application claims the benefit of priority to U.S. Provisional Patent Application No. 63/469,244, filed May 26, 2023.

### BACKGROUND OF THE INVENTION

Urolithins have potent effects on the improvement of a number of health conditions, and they have been shown to be highly biologically active in vitro and in vivo. Urolithins have been proposed as treatments of a variety of conditions including conditions related to inadequate mitochondrial activity, including obesity, memory decline, reduced metabolic rate, metabolic syndrome, diabetes mellitus, cardiovascular disease, hyperlipidemia, neurodegenerative diseases, cognitive disorder, mood disorder, stress, anxiety disorder, fatty liver disease and for improving liver function and weight management. In particular, urolithins have been shown to have beneficial effects in the enhancement of muscle function.

Trigonelline is an alkaloid formed by the methylation of the nitrogen atom of niacin (Vitamin B3). Trigonelline is a product of niacin metabolism that is excreted in urine of mammals. Trigonelline is an important NAD⁺ precursor which feeds into the NAD⁺ pathway. NAD⁺ is an enzyme co-factor that is essential for the function of several enzymes related to reduction-oxidation reactions and energy metabolism. Trigonelline was shown to help to increase NAD⁺ levels in skeletal muscle cells and rodent tissues. It has also been shown to increase lifespan in C. elegans models. (US 2022/0241259 A1). Further, it has been shown to induce muscle mass in rodents (U.S. Pat. No. 9,241,938 B2; US 2011/0142974 A1).

### SUMMARY OF THE INVENTION

One aspect of the invention is a composition comprising a urolithin or a pharmaceutically acceptable salt thereof and trigonelline or a pharmaceutically acceptable salt thereof.

Also provided herein are methods of increasing or maintaining mitochondrial function, increasing or maintaining mitochondrial function, or enhancing or maintaining muscle performance, comprising administering to a subject in need thereof an effective amount of the composition.

### BRIEF DESCRIPTION OF THE DRAWING

FIG. 1 shows the induction of mitochondrial respiration (basal, top) with 50 μM urolithin A, 50 μM trigonelline alone, or a combination of both; and the induction of mitochondrial respiration (maximal, bottom) with 50 μM urolithin A, 50 μM trigonelline alone, or a combination of both.

FIG. 2: shows basal (top) and maximal (bottom) mitochondrial respiration expressed as oxygen consumption rate (OCR, pmol/min), normalized over cellular DNA content and expressed as fold change over DMSO control of the basal respiration experimental condition. Cells were treated as indicated with Urolithin A (UA) 6.25 uM and trigonelline (Trig) 500 uM, alone or in combination. DMSO 0.1% was used as control. Data are expressed as mean+/−SD. One-Way ANOVA, * p.val.<0.05.

### DETAILED DESCRIPTION OF THE INVENTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

As used herein, a “urolithin” refers to a compound of Formula I:

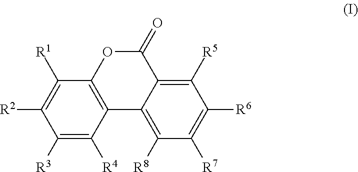

wherein
R¹, R², R³, R⁴, R⁵, R⁶, R⁷, and R⁸ are independently selected from the group consisting of H and OR; and
R is independently for each occurrence H, substituted or unsubstituted alkyl, substituted or unsubstituted aryl, a substituted or unsubstituted monosaccharide, or a substituted or unsubstituted oligosaccharide.

In one embodiment, “urolithin” refers to any one of or a combination of any of urolithin A, urolithin B, urolithin C, and urolithin D.

In another embodiment, “urolithin” refers to any one of or a combination of any of urolithin A.

The compounds of Formula I can be prepared according to procedures described in WO/2015/100213 A2 or WO/2019/168972 A1.

Trigonelline has the structure of Formula II:

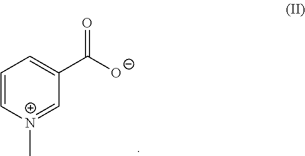

A salt of trigonelline refers to any salt form of trigonelline which can be used to formulate trigonalline into a nutraceutically acceptable form. Examples of suitable trigonelline salts include sulfate, hydrogen sulfate, chloride, phosphate, and citrate. The monohydrate form of trigonelline may also be used in the composition of the present invention.

In certain embodiments, the trigonelline in the composition is a component of or sourced from a coffee bean extract.

In certain embodiments, the trigonelline in the composition is a component of or sourced from fenugreek.

In certain embodiments, the trigonelline in the composition is a component of or sourced from oats.

In certain embodiments, the composition comprises a coffee bean extract.

In certain embodiments, the composition comprises an oat extract.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted, and that the description includes both substituted aryl radicals and aryl radicals having no substitution.

As used herein, the term “treat” as used in connection with a disease, disorder, or condition of a subject, means to reduce by a detectable amount at least one clinical or objective manifestation of the disease, disorder, or condition of a subject. In one embodiment, the term “treat” used in connection with a disease, disorder, or condition of a subject, means to cure the disease, disorder, or condition of a subject.

As used herein, a “food product” refers to a product prepared from a natural food. Non-limiting examples of food products include juices, wines, concentrates, jams, jellies, preserves, pastes, and extracts.

As used herein, a “nutritional supplement” refers to a product suitable for consumption or other administration principally for its health-promoting properties rather than its caloric content.

As used herein, the term “alkyl” refers to a straight chain or branched, noncyclic or cyclic, unsaturated or saturated aliphatic hydrocarbon radical containing carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

As used herein, the term “aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

As used herein, the term “monosaccharide” refers to a simple sugar of the formula (CH2O)n. The monosaccharides can be straight-chain or ring systems, and can include a saccharose unit of the formula —CH(OH)—C(═O)—. Examples of monosaccharides include erythrose, threose, ribose, arabinose, xylose, lyxose, allose, altrose, glucose, mannose, gulose, idose, galactose, talose, erythulose, ribulose, xyulose, psicose, fructose, sorbose, tagatose, erythropentulose, threopentulose, glycerotetrulose, glucopyranose, fructofuranose. In certain embodiments, monosaccharide refers to glucopyranose.

As used herein, the term “oligosaccharide” refers to saccharide consisting of at least two, up to 10 glycosidically linked monosaccharide units, preferably of 2 to 8 monosaccharide units, more preferably of 2 to 7 monosaccharide units, and even more preferably of 2 to 6 monosaccharide units or of 2 to 5 monosaccharide units.

The term “substituted” as used herein (for example, in the context of a substituted alkyl or substituted aryl) means that at least one hydrogen atom is replaced with a substituent. “Substituents” within the context of this invention include halogen, hydroxy, oxo, cyano, nitro, imino, thioxo, amino, alkylamino, dialkylamino, alkyl, alkoxy, alkylthio, haloalkyl, aryl, aralkyl, heteroaryl, heteroarylalkyl, heterocycle and heterocyclealkyl, as well as —NRₐR_{b}, —NRₐC(═O)R_{b}, —NRaC(═O)NRaNRb, —NRₐC(═O)OR_{b}—NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ, —S(═O)₂ORₐ, ═NSO₂Rₐ and —SO₂NRₐR_{b}. In the foregoing, Rₐ and R_{b} in this context may be the same or different and independently hydrogen, alkyl, haloalkyl, cycloalkyl, aryl, aralkyl, heterocyclyl. In addition, the foregoing substituents may be further substituted with one or more of the above substituents.

### Compositions of the Invention

Provided herein is a composition, comprising a urolithin or a pharmaceutically acceptable salt thereof; and trigonelline or a pharmaceutically acceptable salt thereof.

Also provided herein is a composition, comprising a urolithin or a pharmaceutically acceptable salt thereof; and nicotinic acid or a pharmaceutically acceptable salt thereof.

Also provided herein is a composition, comprising a urolithin or a pharmaceutically acceptable salt thereof; trigonelline or a pharmaceutically acceptable salt thereof; and nicotinic acid or a pharmaceutically acceptable salt thereof.

In certain embodiments, the urolithin is urolithin A.

In certain embodiments, the urolithin is urolithin B.

In certain embodiments, the urolithin is urolithin C.

In certain embodiments, the urolithin is urolithin D.

In certain embodiments, the urolithin is selected from the group consisting of urolithin A, urolithin B, urolithin C, urolithin D, and any combination thereof.

In certain embodiments, the urolithin is selected from the group consisting of urolithin A, urolithin B, and a combination of urolithin A and urolithin B.

In certain embodiments, the trigonelline is trigonelline monohydrate.

In certain embodiments, the trigonelline is trigonelline hydrochloride.

In certain embodiments, the composition further comprising a pharmaceutically acceptable carrier.

In certain embodiments, the composition comprises 50 mg-500 mg of trigonelline or a pharmaceutically acceptable salt thereof.

In certain embodiments, the composition comprises 70 mg-1050 mg of the urolithin or pharmaceutically acceptable salt thereof.

In certain embodiments, the composition comprises about 500 mg or about 1000 mg of the urolithin or pharmaceutically acceptable salt thereof.

In certain embodiments, the composition comprises about 140 mg to about 630 mg of the urolithin or pharmaceutically acceptable salt thereof.

In certain embodiments, the composition comprises about 210 mg to about 560 mg of the urolithin or pharmaceutically acceptable salt thereof.

In certain embodiments, the composition comprises about 140 mg to about 525 mg of the urolithin or pharmaceutically acceptable salt thereof.

In certain embodiments, the composition comprises about 280 mg to about 490 mg of the urolithin or pharmaceutically acceptable salt thereof.

In certain embodiments, the composition comprises about 250 mg of the urolithin or pharmaceutically acceptable salt thereof.

In certain embodiments, the composition comprises about 500 mg of the urolithin or pharmaceutically acceptable salt thereof.

In certain embodiments, the composition comprises about 1000 mg of the urolithin or pharmaceutically acceptable salt thereof.

In certain embodiments, the composition comprises about 500 mg to about 2000 mg of the nicotinic acid or pharmaceutically acceptable salt thereof.

In certain embodiments, the composition comprises about 500 mg, about 1000 mg, or about 2000 mg of the nicotinic acid or pharmaceutically acceptable salt thereof.

### METHODS OF THE INVENTION

Provided herein is a method of increasing or maintaining mitochondrial function, comprising administering to a subject in need thereof an effective amount of the composition of the present invention, thereby increasing or maintain mitochondrial function.

In certain embodiments, the mitochondrial function is selected from mitochondrial respiration, mitochondrial activity, and or mitochondrial biogenesis.

In certain embodiments, mitochondrial function is increased, mitochondrial activity is increased, and/or mitochondrial biogenesis is increased.

In certain embodiments, ATP levels are increased in a tissue, e.g. muscle tissue, in the subject.

In certain embodiments, autophagy, e.g., mitophagy, is increased in the subject.

In certain embodiments, Nicotinamide adenine dinucleotide (NAD⁺) production is increased in the subject.

Provided herein is a method of increasing NAD⁺ production, comprising administering to a subject in need thereof an effective amount of the composition of the present invention, thereby increasing NAD⁺ production.

Provided herein is a method of treating, preventing, or managing a mitochondrial-related disease or condition associated with altered mitochondrial function or a reduced mitochondrial density, comprising administering to a subject in need thereof a therapeutically effective amount of the composition of the present invention, thereby treating, preventing, or managing the disease or condition associated with altered mitochondrial function or reduced mitochondrial density.

In certain embodiments, the mitochondria-related disease or condition is selected from obesity, reduced metabolic rate, metabolic syndrome, diabetes mellitus, cardiovascular disease, hyperlipidemia, neurodegenerative disease, cognitive disorder, mood disorder, stress, and anxiety disorder.

In certain embodiments, the mitochondria-related disease or condition is decrease of muscle performance or mental performance, age-related decline in mitochondrial function, or inherited disease.

Provided herein is a method of increasing muscle mass in combination with exercise or decreasing the amount of muscle mass lost during time of lesser muscle activity, comprising administering to a subject in need thereof an effective amount of the composition of the present invention, thereby increasing muscle mass.

In certain embodiments, administration is during resistance training, bodybuilding, or weight training.

Provided herein is a method of enhancing or maintaining muscle performance, comprising administering to a subject in need thereof an effective amount of the composition of the present invention, thereby enhancing muscle performance.

In certain embodiments, the muscle performance is selected from strength, speed, and endurance.

In certain embodiments, the said enhancement is improved muscle function.

In certain embodiments, the said enhancement is improved muscle mass.

In certain embodiments, said method maintains muscle function.

In certain embodiments, said method maintains muscle mass.

In certain embodiments, the subject has cachexia or muscle atrophy.

In certain embodiments, the subject does not have a viral infection.

Provided herein is a method of improving or increasing autophagy, comprising administering to a subject in need thereof an effective amount of the composition of the present invention, thereby improving or increasing autophagy.

In certain embodiments, the autophagy is mitophagy.

In certain embodiments, the subject is an elderly subject.

In certain embodiments, the subject is a healthy subject, e.g., not suffering from cancer or predisposed to developing cancer.

Provided herein is a method of treating, preventing, or managing a disease selected from the group consisting of muscle degenerative disorder, sarcopenia, muscular dystrophy, cardiomyopathy, Ulrich myopathy, obesity, cardiovascular disease, atherosclerosis, and a mitochondria-related disease associated with altered mitochondrial function or a reduced mitochondrial density; or a disease related to a metabolic function; comprising administering to a vertebrate in need thereof an effective amount of the composition of the present invention.

Provided herein is a method of preventing, treating, or managing a condition selected from the group consisting of frailty, muscular atrophy, muscle disuse atrophy, skeletal muscle atrophy or impaired muscle strength associated with aging, and skeletal muscle damage; a mitochondria-related condition associated with altered mitochondrial function or a reduced mitochondrial density; a condition related to a metabolic function; in protecting against muscle damage or injury; in protecting or maintaining cardiac muscle; in improving muscle function, muscle strength, muscle mass, muscle endurance, muscle recovery following exercise; in improving balance or coordination; in improving mitochondrial function; or in weight management; comprising administering to a vertebrate in need thereof an effective amount of the composition of the present invention.

Provided herein is a method of treating a disease or condition selected from the group consisting of sarcopenia, sarcopenia of aging, osteoarthritis, muscular atrophy, muscle disuse atrophy, skeletal muscular atrophy, cardiac deterioration, cardiac deterioration with aging, negative consequences linked to aging; improving activity during aging, reducing age-related cataracts, improving balance or coordination, and frailty, comprising administering to a subject in need thereof a therapeutically effective amount of the composition of the present invention.

In certain embodiments, the composition is administered orally.

In certain embodiments, the composition is administered topically.

In certain embodiments, the subject is a human.

In certain embodiments, the human is elderly.

In certain embodiments, the human is elderly.

In certain embodiments, the urolithin is administered in the form of a tablet or capsule.

In certain embodiments, the urolithin is administered over a period of at least about 1 month (e.g., at least about 2 months, at least about 3 months, at least about 4 months, at least about 5 months, at least about 6 months) In certain embodiments, the human is at least about 40 years old (e.g., at least about 50 years old, at least about 60 years old, at least about 70 years old, at least about 80 years old, at least about 90 years old).

In certain embodiments, the composition is administered at least weekly. In other embodiments, the composition is administered at least daily.

In certain embodiments, the composition is administered in the form of a pharmaceutical.

In certain embodiments, the composition is administered in the form of a medical food.

In certain embodiments, the composition is administered in the form of a functional food.

In certain embodiments, the composition is administered in the form of a food additive.

In certain embodiments, the composition is administered in the form of a dietary supplement.

In certain embodiments, the composition is administered in the form of a food product.

### Formulations and Clinical Use

The composition may be administered, to a subject (e.g., mammal) in a variety of ways. For example, the composition can be administered orally or parenterally. Parenterally includes, without limitation, intravenously, intramuscularly, intraperitoneally, subcutaneously, intra-articularly, intrasynovially, intraocularly, intrathecally, topically, or by inhalation. As such, the form of the urolithin dose can be in a variety of forms, including natural foods, processed foods, natural juices, concentrates and extracts, injectable solutions, microcapsules, nano-capsules, liposomes, plasters, inhalation forms, nose sprays, nose drops, eyedrops, sublingual tablets, and sustained-release preparations.

The compounds of this invention can be provided in isolated form. As used herein, the term “isolated” means substantially removed from other compounds or components with which the compound of interest may otherwise be found, for example, as found in nature. In one embodiment, a compound is isolated when it is essentially completely removed from other compounds or components with which the compound of interest may otherwise be found. In one embodiment, a compound is isolated when it is pure.

The compounds of this invention can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

The compounds of the invention can also be formulated as food additives, food ingredients, functional foods, dietary supplements, medical foods, nutraceuticals, or food supplements.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier, wherein each dosage unit, for example, mL or L, contains a predetermined amount of the composition containing one or more compounds of the present invention.

Implants for sustained release formulations are well-known in the art. Implants are formulated as microspheres; slabs, etc., with biodegradable or non-biodegradable polymers. For example, polymers of lactic acid and/or glycolic acid form an erodible polymer that is well-tolerated by the host. The implant containing the inhibitory compounds may be placed in proximity to a site of interest, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to, be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

For clinical use, the composition is administered in a therapeutically effective amount.

The compounds of the invention can also be formulated as food additives, food ingredients, functional foods, dietary supplements, medical foods, nutraceuticals, or food supplements. In certain embodiments, compounds of the invention can be included into nutraceutical beverages of varying volumes to permit the administration of a daily dose in a convenient format. As a non-limiting example, beverages may deliver effective doses in a final volume ranging from 5 mL to 1,000 mL, delivered as a single dose or multiple doses. In certain embodiments, compositions and methods of the invention are utilized for and in non-human animals. Accordingly, compounds and compositions of the invention may be formulated as veterinary products. Compounds and composition may also be formulated into functional foods for administration to animals, for example, dogs, cats, horses, etc.

Dosing will generally be daily to weekly. In one embodiment, dosing is at least weekly. For example, a subject may receive one dose once weekly, twice weekly, thrice weekly, or every other day. In one embodiment, dosing is at least daily. For example, a subject may receive one or more doses daily.

It is believed that dosing for greatest efficacy in humans involves extended, daily administration. Extended use is contemplated to include use for 1 month, 2 months, 3 months, 4 months, 5 months, 6 months, or even longer.

For clinical use, a urolithin thereof will generally be administered in an amount equal or equivalent to 0.2-2000 milligram (mg) of urolithin per kilogram (kg) of body weight of the subject per day. In one embodiment, the urolithin is administered in a dose equal or equivalent to 2-2000 mg of urolithin per kg body weight of the subject per day. In one embodiment, the urolithin is administered in a dose equal or equivalent to 20-2000 mg of urolithin per kg body weight of the subject per day. In one embodiment, the urolithin is administered in a dose equal or equivalent to 50-2000 mg of urolithin per kg body weight of the subject per day. In one embodiment, the urolithin is administered in a dose equal or equivalent to 100-2000 mg of urolithin per kg body weight of the subject per day. In one embodiment, the urolithin is administered in a dose equal or equivalent to 200-2000 mg of urolithin per kg body weight of the subject per day.

The formulations of urolithin can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

In one embodiment, the urolithin is administered at a concentration in the range from about 0.001 microgram/kg to greater than about 500 mg/kg. For example, the concentration may be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed by the present invention.

In one embodiment, the urolithin is administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

In one embodiment, the urolithin is administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

In another embodiment, the urolithin is administered in concentrations that range from 0.01 micromolar to greater than or equal to 500 micromolar. For example, the dose may be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed by the present invention.

In yet another embodiment, the urolithin is administered at concentrations that range from 0.10 microgram/mL to 500.0 microgram/mL. For example, the concentration may be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL. 250.0 microgram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed by the present invention.

Additional non-limiting, illustrative examples of urolithin doses that may be used are provided in Table 1.

TABLE 1
Urolithin Dosing in Mouse and Human

| Mouse Dose | Human Dose |
|---|---|
| (mg/kg/day) | (mg/kg/day) |
| 5 | 0.36 |
| 10 | 0.73 |
| 15 | 1.09 |
| 20 | 1.46 |
| 30 | 2.19 |
| 40 | 2.91 |
| 50 | 3.64 |
| 60 | 4.37 |
| 70 | 5.10 |
| 80 | 5.33 |
| 90 | 6.56 |
| 100 | 7.29 |
| 110 | 8.01 |
| 120 | 8.74 |
| 130 | 9.47 |
| 140 | 10.20 |
| 150 | 10.93 |
| 160 | 11.66 |
| 170 | 12.38 |
| 180 | 13.11 |
| 190 | 13.84 |
| 200 | 14.57 |
| 210 | 15.30 |
| 220 | 16.03 |
| 230 | 16.76 |
| 240 | 17.48 |
| 250 | 18.21 |
| 260 | 18.94 |
| 270 | 19.67 |
| 280 | 20.40 |
| 290 | 21.13 |
| 300 | 21.86 |
| 310 | 22.58 |
| 320 | 23.31 |
| 330 | 24.04 |
| 340 | 24.77 |
| 350 | 25.50 |
| 360 | 26.23 |
| 370 | 26.96 |
| 380 | 27.68 |
| 390 | 28.41 |
| 400 | 29.14 |
| 410 | 29.87 |
| 420 | 30.60 |
| 430 | 31.33 |

Additional non-limiting, illustrative examples of urolithin dose ranges that may be used are provided in Table 2.

TABLE 2
Urolithin Dosing in Humans
Human Dose Ranges (mg/kg/day)

| Low | High |
|---|---|
| 0.2 | 4 |
| 0.2 | 5 |
| 0.2 | 6 |
| 0.2 | 7 |
| 0.2 | 8 |
| 0.2 | 9 |
| 0.2 | 10 |
| 0.2 | 15 |
| 0.2 | 20 |
| 0.2 | 25 |
| 0.2 | 30 |
| 0.2 | 35 |
| 0.2 | 40 |
| 0.5 | 2 |
| 0.5 | 3 |
| 0.5 | 4 |
| 0.5 | 5 |
| 0.5 | 10 |
| 0.5 | 15 |
| 0.5 | 20 |
| 0.5 | 25 |
| 0.5 | 30 |
| 0.5 | 35 |
| 0.5 | 40 |
| 0.75 | 5 |
| 0.75 | 10 |
| 0.75 | 15 |
| 0.75 | 20 |
| 0.75 | 25 |
| 0.75 | 30 |
| 0.75 | 35 |
| 0.75 | 40 |
| 1 | 2 |
| 1 | 3 |
| 1 | 4 |
| 1 | 5 |
| 1 | 6 |
| 1 | 7 |
| 1 | 8 |
| 1 | 9 |
| 1 | 10 |
| 1 | 11 |
| 1 | 12 |
| 1.25 | 4 |
| 1.25 | 5 |

Any dose may be given as a single dose or as divided doses.

In one embodiment, the urolithin is administered in a dose sufficient to achieve a peak serum level of urolithin and its known metabolites (glucoronides, sulfonates, etc.) of at least 0.001 micromolar (μM). In one embodiment, the urolithin is administered in a dose sufficient to achieve a peak serum level of urolithin of at least 0.01 μM. In one embodiment, the urolithin is administered in a dose sufficient to achieve a peak serum level of urolithin of at least 0.1 μM. In one embodiment, the urolithin is administered in a dose sufficient to achieve a peak serum level of urolithin of at least 1 μM. In various embodiments, the urolithin is administered in a dose sufficient to achieve a peak serum level of urolithin of at least 10 μM, at least 20 μM, at least 30 μM, at least 40 μM, at least 50 μM, at least 60 μM, at least 70 μM, at least 80 μM, at least 90 μM, at least 100 μM, or at least 200 μM.

In one embodiment, the urolithin is administered in a dose sufficient to achieve a sustained serum level of urolithin of at least 0.001 micromolar (μM). In one embodiment, the urolithin is administered in a dose sufficient to achieve a sustained serum level of urolithin of at least 0.01 μM. In one embodiment, the urolithin is administered in a dose sufficient to achieve a sustained serum level of urolithin of at least 0.1 μM. In one embodiment, the urolithin is administered in a dose sufficient to achieve a sustained serum level of urolithin of at least 1 μM. In one embodiment, the urolithin is administered in a dose sufficient to achieve a sustained serum level of urolithin of at least 10 MK, the urolithin is administered in a dose sufficient to achieve a sustained serum level of urolithin of at least 50 μM. The sustained serum level can be measured using any suitable method, for example, high pressure liquid chromatography (HPLC) or HPLC-MS.

In one embodiment, the trigonelline is administered in a dose from 5 to 5,000 mg/day. In other embodiments, the trigonelline is administered in a dose from 10 to 3000 mg/day. In other embodiments, the trigonelline is administered in a dose from 50-500 mg/day.

In certain embodiments, the trigonelline is administered in a dose from 50-100 mg/day, 75-125 mg/day, 100-150 mg/day, 125-175 mg/day, 200-225 mg/day, or 250-300 mg/day.

### EXAMPLES

The invention now being generally described, it will be more readily understood by reference to the following, which is included merely for purposes of illustration of certain aspects and embodiments of the present invention, and is not intended to limit the invention.

### Example 1: Synergistic Effect of Urolithin A and Trigonelline on Mitochondrial Respiration

A combined treatment of urolithin A and trigonelline was performed and mitochondrial respiration was measured in rodent skeletal muscle cells.

### Methods

Fatty acid oxidation analysis on C2C12 myotubes was measured with a Seahorse XF96 extracellular flux analyzer. C2C12s were seeded at 8,000 cells/well in an XF96 cell culture microplate. Once cells reached confluence, they were differentiated with DMEM (Gibco, 31966047) supplemented with 2% Horse serum, 1% PenStrep and 1% Hepes for 5 days. Cells were treated with UA 50 uM and Trigonelline 50 uM (Sigma, T5509) for 24 hours in substrate-limited media (Gibco, A1443001) supplemented with 0.5 mM glucose, 1 mM glutamine, 1% Oleic acid and 0.5 mM L-Carnitine. Maximal respiration was monitored by injection of 3 μM FCCP in the cartridge. Prior to the assay, cells were incubated in a Fatty acid oxidation medium (111 mM NaCl, 4.7 mM KCl, 1.25 mM CaCl₂, 2 mM MgSO₄, 1.2 mM NaH₂PO₄, 5 mM L-Carnitine, 5 mM Hepes, 2.5 mM Glucose) for 20 min, solution must be adjusted to pH 7.4 at 37 °C.

### Results

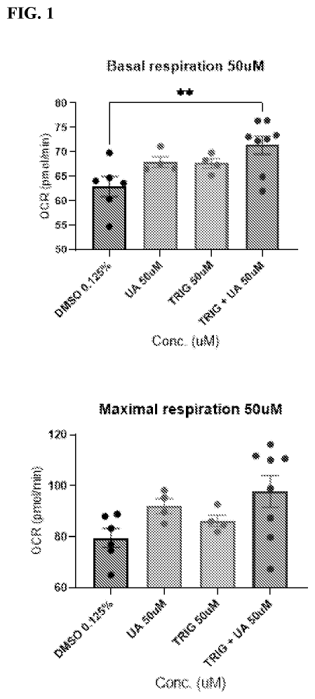

Mouse muscle C2C12 myotubes were treated for 24 hours with either urolithin A alone, trigonelline alone, or a combination of both compounds. Urolithin A alone induced mitochondrial respiration (FIG. 1) as shown previously (Ryu, D. et al. Nature Medicine 2016, 22, 879). Mitochondrial respiration was also increased with trigonelline alone (FIG. 1). This was unexpected since trigonelline at this dose was not shown to have a significant biological effect on NAD production in human cells (see FIG. 1 of US 2022/0241259 A1).

Surprisingly, the combination of both urolithin A (UA) and trigonelline (TRIG) showed a synergistic effect on mitochondrial respiration at basal level (FIG. 1, top), and at the combination also showed an increase at maximal levels (FIG. 1, bottom)

### Example 2: Synergistic Effect of Urolithin A and Trigonelline on Mitochondrial Respiration (Human Primary Skeletal Muscle Myoblasts)

A combined treatment of urolithin A and trigonelline was performed and mitochondrial respiration was measured in human primary skeletal muscle cells.

### Methods

Mitochondrial respiration was measured as oxygen consumption rate on HSMM (LZ-CC-2580, Lonza), human primary skeletal muscle myoblasts was measured with a Seahorse XF96 extracellular flux analyzer. HSMMs were seeded at 3,500 cells/well in an XF96 cell culture microplate and cultured at 37 °C in a humidified atmosphere of 5% C02 in SKM-M Growth medium (AMSbio). The day after seeding cells were treated with UA 6.25 uM and Trigonelline 500 uM (Sigma, T5509) for 24 hours in SKM-M Growth medium. Oxygen consumption rate was measured in Krebs Cycle medium (135 mM NaCl, KCL 3.6 mM, 0.5 mM NaH2PO4, 0.5 mM MgSO4, 10 mM HEPES, 5 mM NaHCO₃) supplemented with 10 mM Glucose, 10 mM pyruvate and 2 mM Glutamine. Maximal respiration was monitored by injection of 6 μM FCCP in the cartridge. To normalize for differences in cell number across well, cellular DNA content was measured in the same microplate using the CyQuant NF kit (Thermofisher, C35006), following the manufacturer's instructions. Normalized mitochondrial respiration was then calculated as ratio between oxygen consumption rate and DNA content and expressed as fold change over the DMSO of the basal condition.

### Results

Human HSMM cells were treated for 24 hours with either urolithin A alone, trigonelline alone, or a combination of both compounds. Oxygen consumption values have been normalized over cellular DNA content. Urolithin A alone induced mitochondrial respiration as shown previously in the C2C12 muscle cell line (Ryu, D. et al. Nature Medicine 2016, 22, 879). Mitochondrial respiration was also increased with trigonelline alone. This was unexpected since trigonelline did show an improvement in mitochondrial respiration in HSMM myotubes, only at a higher dose of 1000 uM, after a longer treatment (72 h) and only by simultaneous blocking of the NAD+ salvage pathway with FK866 (Membrez, M. et al. Nat Metab. 6, 433-447).

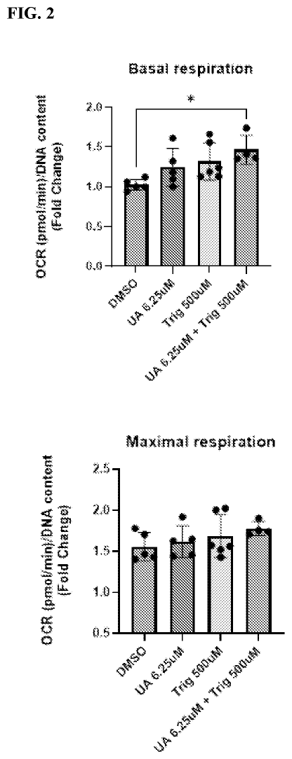

Surprisingly, the combination of both urolithin A (UA) and trigonelline (TRIG) showed a significant synergistic effect (p<0.05) on mitochondrial respiration at basal level (FIG. 2, top), and at the combination also showed an increase at maximal levels (FIG. 2, bottom).

### INCORPORATION BY REFERENCE

All patents and published patent applications mentioned in the description above are incorporated by reference herein in their entirety.

### EQUIVALENTS

Having now described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

We claim:

1. A composition, comprising a urolithin or a pharmaceutically acceptable salt thereof; and trigonelline or a pharmaceutically acceptable salt thereof.

2. The composition of claim 2, wherein the urolithin is urolithin A.

3. The composition of claim 1 or 2, wherein the trigonelline is trigonelline monohydrate.

4. The composition of any one of claims 1-3, further comprising a pharmaceutically acceptable carrier.

5. A method of increasing or maintaining mitochondrial function, comprising administering to a subject in need thereof an effective amount of the composition of any one of claims 1-4, thereby increasing or maintaining mitochondrial function.

6. The method of claim 5, wherein the mitochondrial function is selected from mitochondrial respiration, mitochondrial activity, and or mitochondrial biogenesis.

7. A method of treating, preventing, or managing a mitochondrial-related disease or condition associated with altered mitochondrial function or a reduced mitochondrial density, comprising administering to a subject in need thereof a therapeutically effective amount of the composition of any one of claims 1-4, thereby treating, preventing, or managing the disease or condition associated with altered mitochondrial function or reduced mitochondrial density.

8. The method of claim 7, wherein the mitochondria-related disease or condition is selected from obesity, reduced metabolic rate, metabolic syndrome, diabetes mellitus, cardiovascular disease, hyperlipidemia, neurodegenerative disease, cognitive disorder, mood disorder, stress, and anxiety disorder.

9. A method of increasing muscle mass in combination with exercise or decreasing the amount of muscle mass lost during time of lesser muscle activity, comprising administering to a subject in need thereof an effective amount of the composition of any one of claims 1-4, thereby increasing muscle mass.

10. A method of enhancing or maintaining muscle performance, comprising administering to a subject in need thereof an effective amount of the composition of any one of claims 1-4, thereby enhancing muscle performance.

11. The method of claim 10, wherein the muscle performance is selected from strength, speed, and endurance.

12. The method of claim 10, wherein said enhancement is improved muscle function.

13. The method of claim 10, wherein said enhancement is improved muscle mass.

14. The method of any one of claims 9-13, wherein the subject has cachexia or muscle atrophy.

15. A method of improving or increasing autophagy (e.g., mitophagy), comprising administering to a vertebrate in need thereof an effective amount of the composition of any one of claims 1-4.

16. The method of claim 16, wherein the autophagy is mitophagy.

17. A method of treating, preventing, or managing a disease selected from the group consisting of muscle degenerative disorder, sarcopenia, muscular dystrophy, cardiomyopathy, Ulrich myopathy, obesity, cardiovascular disease, atherosclerosis, and a mitochondria-related disease associated with altered mitochondrial function or a reduced mitochondrial density; or a disease related to a metabolic function; comprising administering to a vertebrate in need thereof an effective amount of the composition of any one of claims 1-4.

18. A method of preventing, treating, or managing a condition selected from the group consisting of frailty, muscular atrophy, muscle disuse atrophy, skeletal muscle atrophy or impaired muscle strength associated with aging, and skeletal muscle damage; a mitochondria-related condition associated with altered mitochondrial function or a reduced mitochondrial density; a condition related to a metabolic function; in protecting against muscle damage or injury; in protecting or maintaining cardiac muscle; in improving muscle function, muscle strength, muscle mass, muscle endurance, muscle recovery following exercise; in improving balance or coordination; in improving mitochondrial function; or in weight management; comprising administering to a vertebrate in need thereof an effective amount of the composition of any one of claims 1-4.

19. A method of treating a disease or condition selected from the group consisting of sarcopenia, sarcopenia of aging, osteoarthritis, muscular atrophy, muscle disuse atrophy, skeletal muscular atrophy, cardiac deterioration, cardiac deterioration with aging, negative consequences linked to aging; improving activity during aging, reducing age-related cataracts, improving balance or coordination, and frailty, comprising administering to a subject in need thereof a therapeutically effective amount of the composition of any one of claims 1-4.

20. The method of any one of claims 1-19, wherein the composition comprises 50 mg-500 mg of trigonelline or a pharmaceutically acceptable salt thereof.

21. The method of any one of claims 1-20, wherein the composition comprises 70 mg-1050 mg of the urolithin or pharmaceutically acceptable salt thereof.

22. The method of claim 21, wherein the composition comprises about 500 mg or about 1000 mg of the urolithin or pharmaceutically acceptable salt thereof.

23. The method of any one of claims 1-22, wherein the composition is administered orally.

24. The method of any one of claims 1-22, wherein the composition is administered topically.

25. The method of any one of claims 1-24, wherein the subject is a human.

26. The method of any one of claims 1-24, wherein the subject is an elderly human.
